(19) United States
(12) Patent Application Publication
(10) Pub. No.: US 2012/0253216 A1
(43) Pub. Date: Oct. 4, 2012

(54) Respiration analysis using acoustic signal trends

(76) Inventors: Yongji Fu, Vancouver, WA (US); Yungkai Kyle Lai, Aliso Viejo, CA (US); Bryan Severt Hallberg, Vancouver, WA (US)

(21) Appl. No.: 13/065,817

(22) Filed: Mar. 30, 2011

(51) Int. Cl. A61B 5/08

(52) U.S. Cl. 600/529

(57) ABSTRACT

The present invention isolates respiration phases in an acoustic signal using trend analysis. Once respiration phases are isolated, they are used to estimate respiration parameters. An exemplary method comprises receiving an acoustic signal recording body sounds; identifying candidate peaks at maxima of the signal; identifying candidate valleys at minima of the signal; selecting significant peaks from among the candidate peaks using heights of the candidate peaks; selecting significant valleys from among the candidate valleys using heights of the candidate valleys; detecting silent phases in the signal based at least in part on rise rates from the significant valleys; isolating respiration phases in the signal based at least in part on the significant valleys and the silent phases; calculating respiration parameter estimates based at least in part on the respiration phases; and outputting the respiration parameter estimates.

### BACKGROUND OF THE INVENTION

The present invention relates to physiological monitoring and, more particularly, respiration monitoring through analysis of an acoustic signal.

Monitoring of respiration parameters is crucial in evaluating and predicting the health status of human subjects suffering from pulmonary diseases as well as in other applications. Respiration in humans is typically characterized by two main phases: inspiration, or the intake of air into the lungs, and expiration, or the expelling of air from the lungs. In some cases, silent phase may also be included in which there is barely any air flow. A high respiration rate (i.e., low respiration cycle time), low fractional inspiration time (i.e., inspiration phase time divided by respiration cycle time) or low inspiration to expiration time ratio (i.e., inspiration phase time divided by expiratory phase time, also known as I:E ratio) may indicate obstruction of a subject's airways. A high fractional inspiration time or I:E ratio may provide other information about the status of a monitored subject, for example, may indicate that the subject is currently snoring or speaking. The trend in respiration rate and I:E ratio may also be instructive in some applications.

A common technique for monitoring respiration parameters is lung sound analysis, sometimes called auscultation. The lung sound analysis method has become increasingly popular due in part to the low cost and ready availability of lung sound detection systems. In the lung sound method, a body mounted sound transducer captures lung sounds and generates an acoustic signal recording the lung sounds. The sound transducer is typically placed over the suprastemal notch or at the lateral neck near the pharynx because lung sounds captured in that region typically have a high signal-to-noise ratio and a high sensitivity to variation in flow. Once the acoustic signal with recorded lung sounds has been generated, respiration phases are isolated within the acoustic signal and respiration parameter estimates (e.g., respiration rate, I:E ratio) are calculated.

Known techniques for isolating respiration phases within an acoustic signal often rely heavily on peak analysis. For example, some phase isolation methods identify peak amplitudes in an acoustic signal, and then mark times when rising amplitudes reach a certain percentage of the peaks (e.g., 10%) as the boundary between respiration phases. Unfortunately, these methods are unreliable when the acoustic signal is generated the presence of background noise or other body sounds (e.g., heart sounds) that introduce significant error into amplitude measurements. Moreover, these methods often misidentify respiratory phase boundaries by failing to properly analyze silent phases present in acoustic signals recording the lung sounds of human subjects.

### SUMMARY OF THE INVENTION

The present invention, in a basic feature, isolates respiration phases in an acoustic signal using signal energy envelope trends. Once respiration phases are isolated, they are used to estimate respiration parameters, such as respiration rate and I/E ratio.

In one aspect of the invention, a method for processing an acoustic signal comprises the steps of receiving by a respiration monitoring system an acoustic signal recording body sounds; identifying by the system candidate peaks at maxima of the signal; identifying by the system candidate valleys at minima of the signal; selecting by the system significant peaks from among the candidate peaks using heights of the candidate peaks; selecting by the system significant valleys from among the candidate valleys using heights of the candidate valleys; detecting by the system silent phases in the signal based at least in part on rise rates from the significant valleys; isolating by the system respiration phases in the signal based at least in part on the significant valleys and the silent phases; calculating by the system respiration parameter estimates based at least in part on the respiration phases; and outputting by the system the respiration parameter estimates.

In some embodiments, the method further comprises the step of identifying by the system a true silent phase among the silent phases based at least in part on a respiration phase sequence exhibited by the signal.

In some embodiments, the method further comprises the step of identifying by the system a silent expiration phase among the silent phases based at least in part on a respiration phase sequence exhibited by the signal.

In some embodiments, the method further comprises the step of eliminating by the system redundant peaks from the significant peaks based at least in part on heights of consecutive significant peaks that are uninterrupted by a significant valley.

In some embodiments, the method further comprises the step of eliminating by the system redundant valleys from the significant valleys based at least in part on heights of consecutive significant valleys that are uninterrupted by a significant peak.

In some embodiments, the step of selecting significant peaks comprises selecting candidate peaks having heights that are above zero by at least a first predetermined amount and above heights of immediately preceding significant valleys by at least a second predetermined amount.

In some embodiments, the step of selecting significant valleys comprises selecting candidate valleys having heights that are above zero by less than a first predetermined amount and below heights of immediately preceding significant peaks by at least a second predetermined amount.

In some embodiments, the isolating step comprises designating a period bounded between consecutive significant valleys as a respiration phase.

In some embodiments, the isolating step comprises designating a period bounded between an end of a silent phase and a next significant valley as a respiration phase.

In some embodiments, the monitoring system is a portable ambulatory monitoring device.

In another aspect of the invention, a respiration monitoring system comprises a sound capture system adapted to acquire an acoustic signal recording body sounds; an acoustic signal processing system communicatively coupled with the capture system and adapted to identify candidate peaks at maxima of the signal, identify candidate valleys at minima of the signal, select significant peaks from among the candidate peaks using heights of the candidate peaks, select significant valleys from among the candidate valleys using heights of the candidate valleys, detect silent phases in the signal based at least in part on rise rates from the significant valleys, isolate respiration phases in the signal based at least in part on the significant valleys and the silent phases and calculate respiration parameter estimates based at least in part on the respiration phases; and a data output system communicatively coupled with the processing system and adapted to output the respiration parameter estimates.

In yet another aspect of the invention, an acoustic signal processing system comprises a respiration phase detector adapted to receive an acoustic signal recording body sounds, identify candidate peaks at maxima of the signal, identify candidate valleys at minima of the signal, select significant peaks from among the candidate peaks using heights of the candidate peaks, select significant valleys from among the candidate valleys using heights of the candidate valleys, detect silent phases in the signal based at least in part on rise rates from the significant valleys and isolate respiration phases in the signal based at least in part on the significant valleys and the silent phases; and a respiration parameter calculator communicatively coupled with the respiration phase detector and adapted to receive the signal and respiration phase information, calculate respiration parameter estimates based at least in part on the signal and respiration phase information and output the respiration phase parameter estimates.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows an acoustic signal exhibiting a repetitive respiration cycle of a first class.

FIG. 2 shows an acoustic signal exhibiting a repetitive respiration cycle of a second class.

FIG. 3 shows an acoustic signal exhibiting a repetitive respiration cycle of a third class.

FIG. 4 shows a respiration monitoring system in some embodiments of the invention.

FIG. 5 shows an exemplary raw acoustic signal.

FIG. 6 shows an exemplary acoustic signal after application of a band-pass filter to the signal.

FIG. 7 shows an exemplary acoustic signal energy envelope after application of an envelope detector and smoothing module to the signal.

FIG. 8 shows a method for isolating respiration phases in an acoustic signal in some embodiments of the invention.

FIG. 9 shows use of signal maxima and minima to identify a candidate peak and valley within an acoustic signal in some embodiments of the invention.

FIG. 10 shows use of signal heights to select a significant peak and valley within an acoustic signal in some embodiments of the invention.

FIG. 11 shows use of a signal rise rate to identify a silent phase within an acoustic signal in some embodiments of the invention.

FIG. 12 shows use of signal heights of consecutive significant peaks that are uninterrupted by a significant valley to eliminate a redundant peak in some embodiments of the invention.

FIG. 13 shows use of signal heights of consecutive significant valleys that are uninterrupted by a significant peak to eliminate a redundant valley in some embodiments of the invention.

### DETAILED DESCRIPTION OF A PREFERRED EMBODIMENT

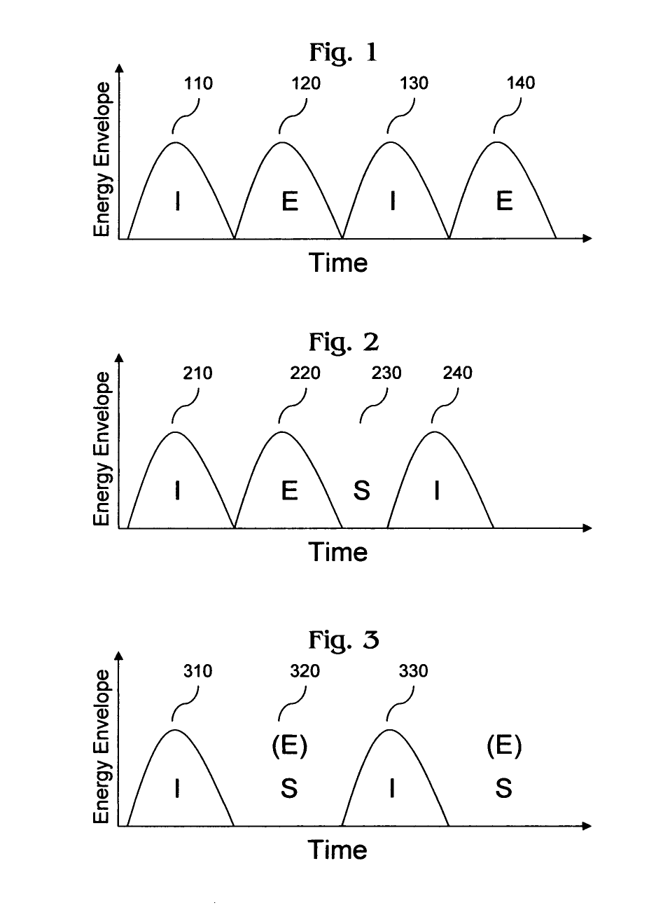

Empirical study shows that human respiration manifested in an acoustic signal exhibits one of three distinct patterns, which can be subject dependent and even vary for the same subject due to many factors such as activities levels and disease status. FIG. 1 shows an acoustic signal energy envelope exhibiting a respiration phase sequence of the first class. This Class I sequence consists of an inspiration phase 110 followed immediately by an expiration phase 120. Expiration phase 120 is followed immediately by the next respiration cycle which again consists of an inspiration phase 130 and an expiration phase 140. There is no silent phase of any significance.

FIG. 2 shows an acoustic signal energy envelope exhibiting a respiration phase sequence of the second class. This Class II sequence consists of an inspiration phase 210 followed immediately by an expiration phase 220, after which comes a brief silent phase 230. This brief silent phase 230 is attributable to a temporary lack of significant respiratory airflow. Accordingly, it is a true silent phase that occurs between expiration phase 220 and the start of the next inspiration phase 240.

FIG. 3 shows an acoustic signal energy envelope exhibiting a respiration phase sequence of the third class. This Class III sequence consists of an inspiration phase 310 followed immediately by an extended silent phase 320. This extended silent phase 320 is attributable to expiration not being loud enough to be detected. Accordingly, extended silent phase 320 is in reality an expiration phase that occurs between inspiration phase 310 and the next inspiration phase 330.

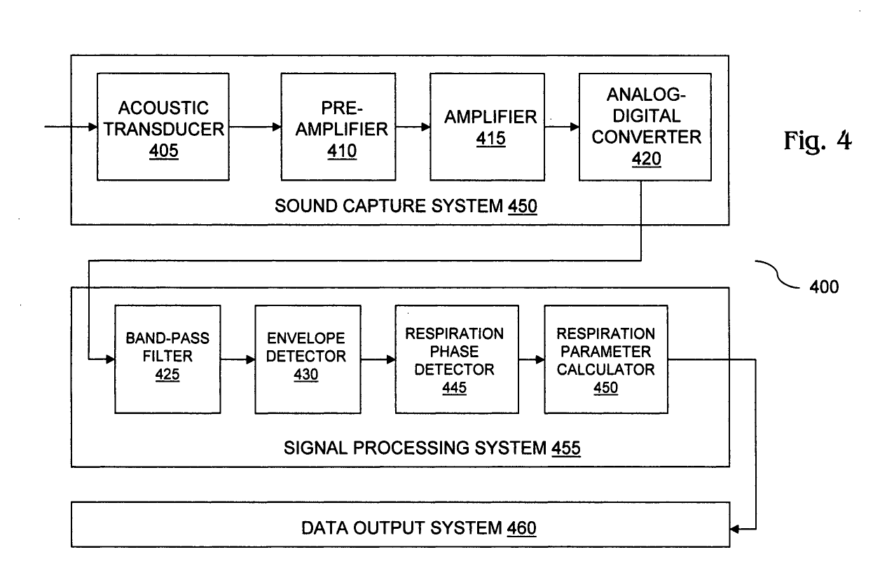

Turning to FIG. 4, a respiration monitoring system 400 adapted to isolate respiration phases in an acoustic signal using trend and silent phase detection and analysis is shown in some embodiments of the invention. Monitoring system 400 includes a sound capture system 450, a signal processing system 455 and a data output system 460 communicatively coupled in series. Monitoring system 400 continually acquires and processes an acoustic signal recording lung sounds and continually outputs respiration parameter estimates based on the acoustic signal. Acoustic signal processing includes isolating respiration phases in the acoustic signal using trend and silent phase detection and analysis, which enables reliable estimation of respiration parameters.

In some embodiments, monitoring system 400 is a portable ambulatory monitoring device that monitors a human subject's respiratory health in real-time as the person performs daily activities. In other embodiments, capture system 450, processing system 455 and output system 460 may be part of separate devices that are remotely coupled via wired or wireless data communication links.

Capture system 450 includes an acoustic transducer 405, a pre-amplifier 410, an amplifier 415 and an analog-to-digital (A/D) converter 420 communicatively coupled in series. Transducer 405 is positioned on the body, such as the trachea or chest, of a human subject being monitored and detects body sounds. Transducer 405 provides high sensitivity, a high signal-to-noise ratio and a generally flat frequency response in the band for respiration sounds. Transducer 405 in some embodiments comprises an omni-directional piezo ceramic microphone housed in an air chamber of suitable depth and diameter. Transducer 405 outputs to pre-amplifier 410 a raw acoustic signal recording body sounds as an analog voltage. Pre-amplifier 410 provides impedance match for the raw acoustic signal received from transducer 405 and amplifies the raw acoustic signal. Amplifier 415 further amplifies the raw acoustic signal received from amplifier 110. ND converter 420 performs ND conversion on the raw acoustic signal received from amplifier 415 and transmits the raw acoustic signal to signal processing system 455 for analysis.

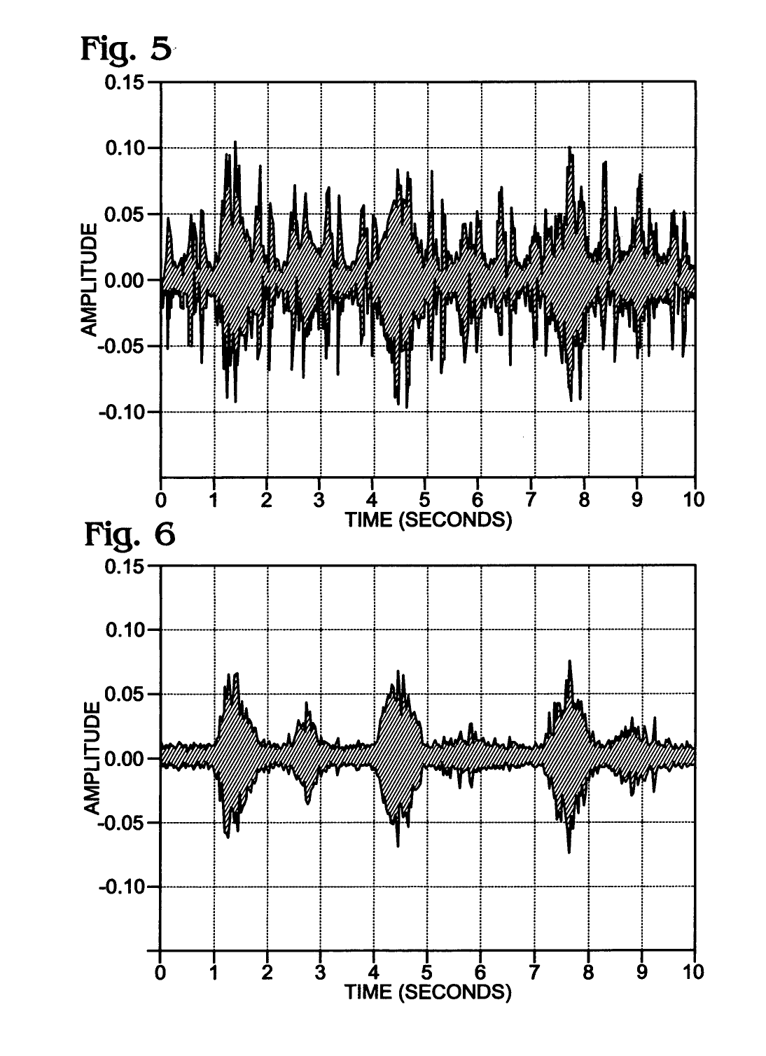

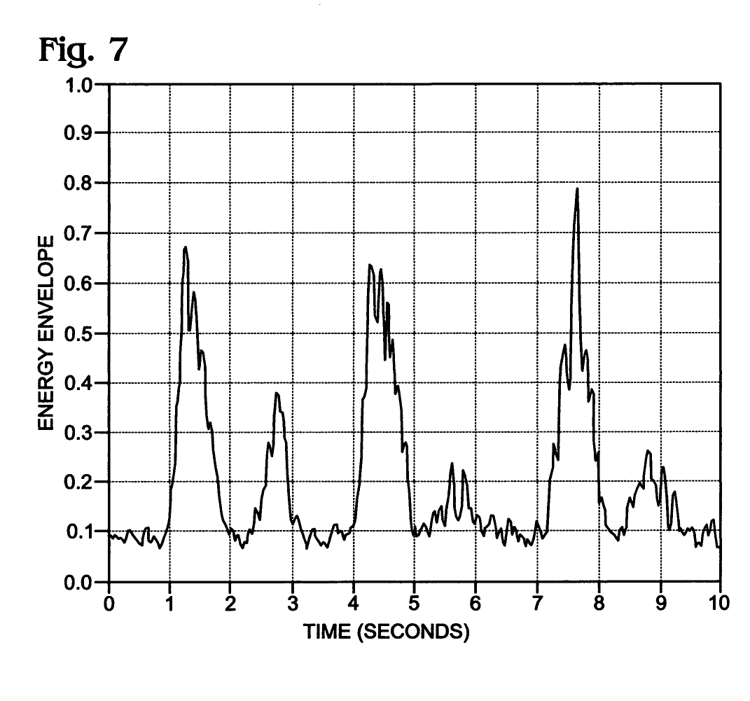

Processing system 455 includes a band-pass filter 425, an envelope detector 430, a respiration phase detector 435 and a respiration parameter calculator 440 communicatively coupled in series. In some embodiments, elements 425, 430, 435, 440 are implemented using software executing under control of a processor. In other embodiments, one or more of elements 430, 435, 440 may be implemented in custom logic or a combination of software and custom logic. Band-pass filter 425 receives a raw acoustic signal from capture system 450. An exemplary raw acoustic signal is shown in FIG. 5. The raw acoustic signal is noisy and heart sounds are intermingled with lung sounds. Band-pass filter 425 applies a high-pass cutoff frequency and a low-pass cutoff frequency to the acoustic signal to isolate the lung sounds. An exemplary resulting signal is shown in FIG. 6. The pulse sequence has been largely removed and the respiratory sequence is better defined due to noise reduction. Next, envelope detector 430 is applied to the acoustic signal to generate a smooth acoustic signal energy envelope. In some embodiments, detector 430 has a smoothing module that applies to the detected signal energy envelope a smooth FIR filter. An exemplary resulting envelope is shown in FIG. 7. This envelope is passed to respiration phase detector 445, which isolates respiration phases in the envelope using trend and silent phase detection and analysis.

In some embodiments, processing system 455 further includes a noisy segment detection and isolation module that detects and isolates particularly noisy segments in the raw acoustic signal prior to application of band-pass filter 425. These noisy segments are excluded from consideration when isolating respiration phases and calculating respiration parameter estimates.

Moreover, in some embodiments, an additional low-pass filter is applied to the signal energy envelope before passing the envelope to respiration phase detector 445 in order to further remove relatively fast-changing non-respiration sounds (e.g., heart sounds). This additional low-pass filter may apply an adaptive cutoff frequency over several iterations and select a cutoff frequency that strikes an appropriate balance between removal of non-respiration sounds and retention of lung sounds for the particular human subject being monitored.

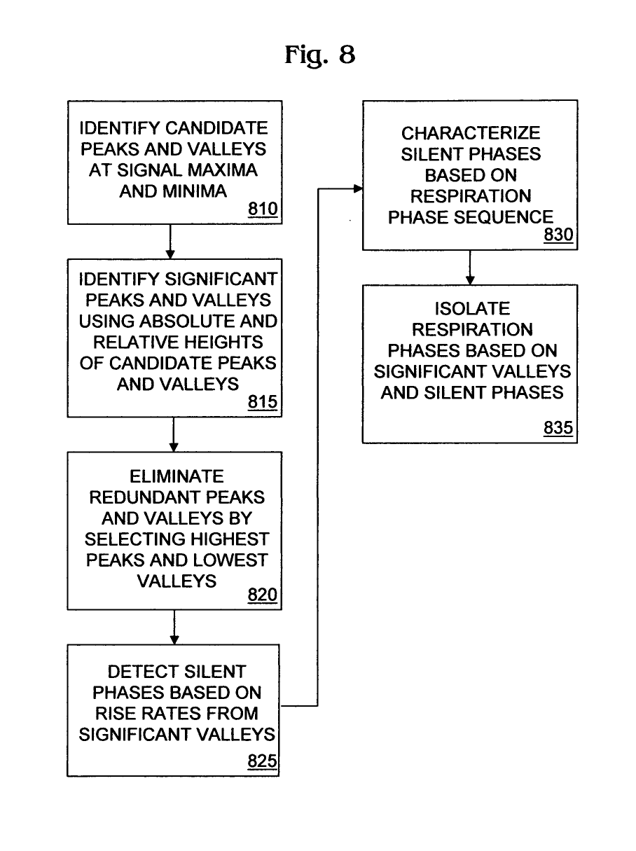

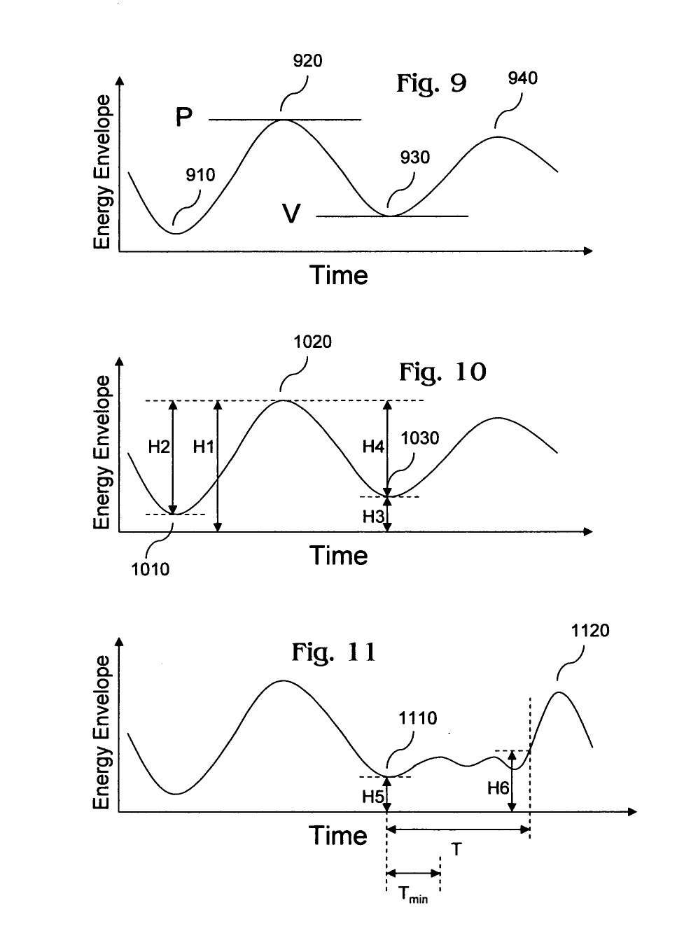

Referring now to FIG. 8, a method performed by respiration phase detector 445 under processor control for isolating respiration phases in an acoustic signal is shown in some embodiments of the invention. The method is applied to an acoustic signal energy envelope such as the exemplary envelope shown in FIG. 7, and will now be described in conjunction with the illustrative diagrams of FIGS. 9-13.

First, phase detector 445 identifies candidate peaks and valleys at signal maxima and minima (810). Phase detector 445 marks all times when the signal reaches a maximum, as indicated by the signal slope (derivative) falling from a positive value to zero, as candidate peaks. Similarly, phase detector 445 marks all times when the signal reaches a minimum, as indicated by the signal slope (derivative) rising from a negative value to zero, as candidate valleys. For example, in FIG. 9, an acoustic signal energy envelope is shown to have a first candidate valley 910, followed by a first candidate peak 920, followed by a second candidate valley 930, followed by a second candidate peak 940.

Next, phase detector 445 selects significant peaks and valleys from among the candidate peaks and valleys using absolute and relative heights of the candidate peaks and valleys (815). Significant peak and valley selection may be better understood by reference to FIG. 10. There, an acoustic signal energy envelope is shown to have a candidate peak 1020 followed by a candidate valley 1030. Phase detector 445 performs a first check to verify that the absolute height (H1) of candidate peak 1020, that is, the amount by which candidate peak 1020 is above zero, exceeds a minimum absolute height threshold. Phase detector 445 performs a second check to verify that the relative height (H2) of candidate peak 1020, that is, the amount by which candidate peak 1020 is above the immediately preceding significant valley 1010, exceeds a minimum relative height threshold. If candidate peak 1020 passes both checks, phase detector 445 selects candidate peak 1020 as significant; otherwise, phase detector 445 disregards candidate peak 1020. Next, phase detector 445 performs a first check to verify that the absolute height (H3) of candidate valley 1030, that is, the amount by which candidate valley 1030 is above zero, does not exceed a maximum absolute height threshold. Phase detector 445 performs a second check to verify that the relative height (H4) of candidate valley 1030, that is, the amount by which candidate valley 1030 is below the immediately preceding significant peak 1020, exceeds a minimum relative height threshold. If candidate valley 1030 passes both checks, phase detector 445 selects candidate valley 1030 as significant; otherwise, phase detector 445 disregards candidate valley 1030.

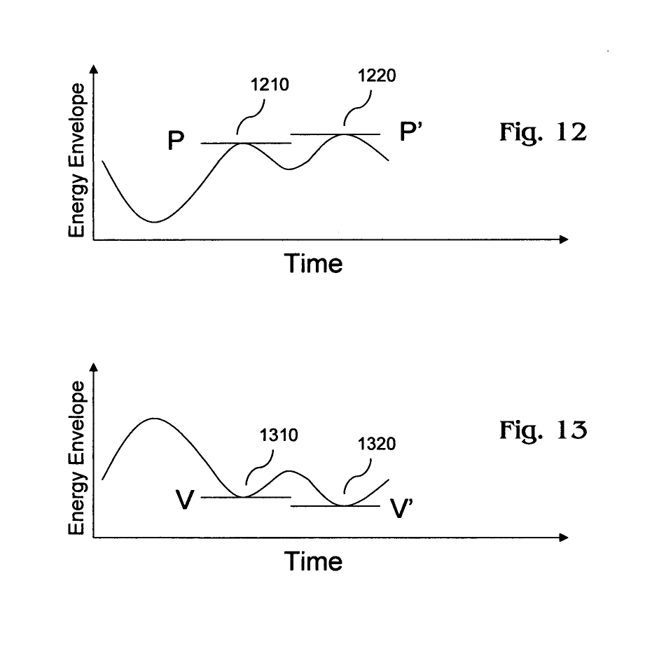

Next, phase detector 445 eliminates redundant peaks and valleys by selecting the highest peaks and lowest valleys (820). Due to background noise, heart sound artifacts or other factors causing signal distortion, the selection of Step 815 may yield two or more significant peaks that are uninterrupted by a significant valley, and/or may yield two or more significant valleys that are uninterrupted by a significant peak. For example, in FIG. 12, a first significant peak 1210 is followed by a second significant peak 1220 without a significant valley separating peaks 1210, 1220. Accordingly, phase detector 445 disregards the lower significant peak 1210 among the two significant peaks 1210, 1220 as being redundant. Similarly, in FIG. 13, a first significant valley 1310 is followed by a second significant valley 1320 without a significant peak separating valleys 1310, 1320. Accordingly, phase detector 445 disregards the higher significant valley 1310 among the two significant valleys 1310, 1320 as being redundant.

Next, phase detector 445 detects silent phases based on rise rates from significant valleys (825). As described earlier in conjunction with FIGS. 2 and 3, the Class II and Class III respiration phase sequences exhibit silent phases, which can be true silent phases attributable to the lack of meaningful airflow (for Class II) or silent expiration phases attributable to expiration not being sufficiently loud to be detected (for Class III). These silent phases are accounted for in order to reliably isolate respiration phases and reliably estimate respiration parameters. More particularly, the rise rate from each significant valley is determined and a silent phase is identified where the rise rate is below a rise rate threshold after minimum period. In FIG. 11, for example, a significant valley 1110 is followed by a significant peak 1120. Phase detector 445 begins measuring the rise rate from significant valley 1110 after a minimum period Tₘᵢₙ and determines that the rise rate does not exceed the rise rate threshold until after a period T, at which point the rise rate is characterized by (H6−H5)/T. Accordingly, phase detector 445 designates the period T as a silent phase.

Next, phase detector 445 characterizes silent phases as true silent phases or silent expiration phases based on a respiration phase sequence exhibited by the envelope (830). For example, if a silent phase detected in the envelope follows two consecutive non-silent phases, the Class II sequence (see FIG. 2) is presumed and the silent phase is designated a true silent phase. On the other hand, if a silent phase detected in the envelope follows a non-silent phase that was immediately preceded by a silent phase, the Class III sequence (see FIG. 3) is presumed and the silent phase is designated a silent expiration phase. The length of a silent phase may be used as an additional or alternative criterion in characterizing a silent phase, as true silent phases tend to be of shorter duration than silent expiration phases.

Next, phase detector 445 isolates respiration phases based on significant valleys and silent phases (835). Each period bounded between consecutive significant valleys without any interrupting silent phase is designated a respiration phase. Each period bounded between the end of a silent phase and the next significant valley is designated a respiration phase. And, naturally, each silent expiration phase is designated a respiration phase. Phase detector 445 then passes the envelope with isolated respiration phases to respiration parameter calculator 450.

Calculator 450 generates estimates of one or more respiration parameters for the subject being monitored using the envelope and isolated respiration phases. Monitored respiration parameters may include, for example, respiration rate, fractional inspiration time and/or inspiration to expiration time ratio. Where the respiration phase sequence does not permit inspiration and expiration phases to be readily distinguished, a known technique, such as requiring the subject to explicitly identify an initial inspiration phase, may be invoked to enable inspiration and expiration phases to be differentiated. Calculator 450 transmits the respiration parameter estimates to data output system 460 for outputting.

In some embodiments, output system 460 has a display screen for displaying respiration data determined using respiration parameter estimates received from processing system 455. In some embodiments, output system 460 in addition to or in lieu of a display screen has an interface to an internal or external data management system that stores respiration data determined using respiration parameter estimates received from processing system 455, and/or an interface that transmits respiration data determined using respiration parameter estimates received from processing system 455 to a remote monitoring device, such as a monitoring device at a clinician facility. Respiration data outputted by output system 460 may include the respiration parameter estimates received from processing system 455 and/or respiration data derived from such physiological parameter estimates.

It will be appreciated by those of ordinary skill in the art that the invention can be embodied in other specific forms without departing from the spirit or essential character hereof. The present description is thus considered in all respects to be illustrative and not restrictive. The scope of the invention is indicated by the appended claims, and all changes that come with in the meaning and range of equivalents thereof are intended to be embraced therein.

What is claimed is:

1. A method for processing an acoustic signal, comprising the steps of:
receiving by a respiration monitoring system an acoustic signal recording body sounds;
identifying by the system candidate peaks at maxima of the signal;
identifying by the system candidate valleys at minima of the signal;
selecting by the system significant peaks from among the candidate peaks using heights of the candidate peaks;
selecting by the system significant valleys from among the candidate valleys using heights of the candidate valleys;
detecting by the system silent phases in the signal based at least in part on rise rates from the significant valleys;
isolating by the system respiration phases in the signal based at least in part on the significant valleys and the silent phases;
calculating by the system respiration parameter estimates based at least in part on the respiration phases; and
outputting by the system the respiration parameter estimates.

2. The method of claim 1, further comprising the step of identifying by the system a true silent phase among the silent phases based at least in part on a respiration phase sequence exhibited by the signal.

3. The method of claim 1, further comprising the step of identifying by the system a silent expiration phase among the silent phases based at least in part on a respiration phase sequence exhibited by the signal.

4. The method of claim 1, further comprising the step of eliminating by the system redundant peaks from the significant peaks based at least in part on heights of consecutive significant peaks that are uninterrupted by a significant valley.

5. The method of claim 1, further comprising the step of eliminating by the system redundant valleys from the significant valleys based at least in part on heights of consecutive significant valleys that are uninterrupted by a significant peak.

6. The method of claim 1, wherein the step of selecting significant peaks comprises selecting candidate peaks having heights that are above zero by at least a first predetermined amount and above heights of immediately preceding significant valleys by at least a second predetermined amount.

7. The method of claim 1, wherein the step of selecting significant valleys comprises selecting candidate valleys having heights that are above zero by less than a first predetermined amount and below heights of immediately preceding significant peaks by at least a second predetermined amount.

8. The method of claim 1, wherein the isolating step comprises designating a period bounded between consecutive significant valleys as a respiration phase.

9. The method of claim 1, wherein the isolating step comprises designating a period bounded between an end of a silent phase and a next significant valley as a respiration phase.

10. The method of claim 1, wherein the monitoring system is a portable ambulatory monitoring device.

11. A respiration monitoring system, comprising:
a sound capture system adapted to acquire an acoustic signal recording body sounds;
an acoustic signal processing system communicatively coupled with the capture system and adapted to identify candidate peaks at maxima of the signal, identify candidate valleys at minima of the signal, select significant peaks from among the candidate peaks using heights of the candidate peaks, select significant valleys from among the candidate valleys using heights of the candidate valleys, detect silent phases in the signal based at least in part on rise rates from the significant valleys, isolate respiration phases in the signal based at least in part on the significant valleys and the silent phases and calculate respiration parameter estimates based at least in part on the respiration phases; and
a data output system communicatively coupled with the processing system and adapted to output the respiration parameter estimates.

12. The monitoring system of claim 11, wherein the processing system is adapted to identify a true silent phase among the silent phases based at least in part on a respiration phase sequence exhibited by the signal.

13. The monitoring system of claim 11, wherein the processing system is adapted to identify a silent expiration phase among the silent phases based at least in part on a respiration phase sequence exhibited by the signal.

14. The monitoring system of claim 11, wherein the processing system is adapted to eliminate redundant peaks from the significant peaks based at least in part on heights of consecutive significant peaks that are uninterrupted by a significant valley.

15. The monitoring system of claim 11, wherein the processing system is adapted to eliminate redundant valleys from the significant valleys based at least in part on heights of consecutive significant valleys that are uninterrupted by a significant peak.

16. An acoustic signal processing system, comprising:
a respiration phase detector adapted to receive an acoustic signal recording body sounds, identify candidate peaks at maxima of the signal, identify candidate valleys at minima of the signal, select significant peaks from among the candidate peaks using heights of the candidate peaks, select significant valleys from among the candidate valleys using heights of the candidate valleys, detect silent phases in the signal based at least in part on rise rates from the significant valleys and isolate respiration phases in the signal based at least in part on the significant valleys and the silent phases; and
a respiration parameter calculator communicatively coupled with the respiration phase detector and adapted to receive the signal and respiration phase information, calculate respiration parameter estimates based at least in part on the signal and respiration phase information and output the respiration phase parameter estimates.

17. The processing system of claim 16, wherein the phase detector is adapted to identify a true silent phase among the silent phases based at least in part on a respiration phase sequence exhibited by the signal.

18. The processing system of claim 16, wherein the phase detector is adapted to identify a silent expiration phase among the silent phases based at least in part on a respiration phase sequence exhibited by the signal.

19. The processing system of claim 16, wherein the phase detector is adapted to eliminate redundant peaks from the significant peaks based at least in part on heights of consecutive significant peaks that are uninterrupted by a significant valley.

20. The processing system of claim 16, wherein the phase detector is adapted to eliminate redundant valleys from the significant valleys based at least in part on heights of consecutive significant valleys that are uninterrupted by a significant peak.
